(19) United States
(12) Patent Application Publication
(10) Pub. No.: US 2023/0017173 A1
(43) Pub. Date: Jan. 19, 2023

(54) BREAST ULTRASOUND SCREENING AND DIAGNOSTICS SYSTEM AND METHOD

(71) Applicant: SonoCine, Inc., Reno, NV (US)

(72) Inventor: Rafael Carballido Quintas, Weston, FL (US)

(21) Appl. No.: 17/866,125

(22) Filed: Jul. 15, 2022

Related U.S. Application Data
(60) Provisional application No. 63/222,354, filed on Jul. 15, 2021.
(60) Provisional application No. 63/252,356, filed on Oct. 5, 2021.

(51) Int. Cl. A61B 8/08; A61B 8/00

(52) U.S. Cl. CPC A61B 8/0825; A61B 8/085; A61B 8/488; A61B 8/5207; A61B 8/543

(57) ABSTRACT

A system for screening and diagnostics of cellular tissue includes a system controller, an ultrasound apparatus, an electromechanical positioning apparatus, and an image analyzer communicably coupled together. The electromechanical positioning apparatus includes an articulated arm, and the ultrasound apparatus includes a scan head coupled to an end of the articulated arm. The system controller controls the electromechanical positioning apparatus to move the scan head adjacent the cellular tissue while controlling the ultrasound apparatus to generate a first set of ultrasound images of the cellular tissue. The image analyzer analyzes the ultrasound images, and in response to identifying a potential abnormality, the electromechanical positioning apparatus is controlled to move the scan head to a position adjacent the location of the potential abnormality, where the electromechanical positioning apparatus and the ultrasound apparatus are controlled to generate a second set of ultrasound images of the potential abnormality.

### PRIORITY

Priority is claimed to U.S. Provisional patent application Ser. No. 63/222,354, filed Jul. 15, 2021 and to U.S. Provisional patent application Ser. No. 63/252,356, filed Oct. 5, 2021, the disclosures of which are incorporated herein by reference in their entirety.

### CROSS REFERENCE

The following documents are incorporated herein by reference as if set forth herein in their entirety:

U.S. Pat. No. 6,524,246, filed Oct. 13, 2000 and entitled “Ultrasonic Cellular Tissue Screening System”;

U.S. Patent Publication No. 20070073149, issued as U.S. Pat. No. 8,911,370, filed Nov. 3, 2006 and entitled “Ultrasonic Cellular Tissue Screening System”;

U.S. Patent Publication No. 20150265243, issued as U.S. Pat. No. 10,603,010, filed Mar. 23, 2015, and entitled “System and Method for Performing an Ultrasound Scan of Cellular Tissue”; and

U.S. Patent Publication No. 20190167231, now abandoned, filed Nov. 30, 2018 and entitled “System and Method for Ultrasonic Tissue Screening”.

### FIELD OF THE INVENTION

The field of the present invention is systems and methods for ultrasonic screening and diagnostics of cellular tissue, in particular systems and methods which use machine learning to enhance screening and diagnostics of potential abnormalities within cellular tissue.

### BACKGROUND OF THE INVENTION

Ultrasound as an independent screening tool for diagnosing cancers, particularly in breast tissue, is becoming more widely recognized and accepted within the medical community. Historically, in the context of breast exams and breast cancers, ultrasound was first used to diagnose breast cancers if the location of the abnormality is first discovered by another modality, such as mammography or physical examination. Even today, handheld ultrasound is frequently used as an adjunct screening tool in order to further confirm the nature of an abnormality discovered through use of another modality. Handheld ultrasound is generally not used as a stand-alone screening tool because of the difficulty in achieving complete coverage of the breast tissue and the difficulty of finding abnormalities within the tissue that are not already large enough in size to be found by mammography or physical examination.

Recently, ultrasound as a stand-alone screening tool for breast cancers has become more widely accepted based on the development of technology that enables ultrasound to be used to screen the entire breast tissue and have an acceptably high success rate in finding abnormalities that may be cancerous.

3D automated breast ultrasound screening has been introduced to the market in an attempt to overcome the shortcomings of handled ultrasound and to as a diagnostic tool that could eventually take the place of mammograms. However, most of these 3D systems have significant drawbacks, one of which is the inability to assess the axillary region and tail of the breast tissue. This drawback results in a lack of information about the lymph nodes in that region, and therefore a lack of screening or diagnosis. The 3D automated systems also suffer the drawback that radiologists are required to learn new techniques for reviewing the 3D ultrasound images in order to perform the screening and diagnosis, and this has resulted in reduced interest for these systems within the medical community marketplace

One system that has been developed to uniformly and reliably screen breast tissue for cancer is described in U.S. Pat. No. 6,524,246. This system is an automated whole breast ultrasound screening system that moves an ultrasound probe along the entirety of the breast tissue, including the axillary and tail, to generate a set of ultrasound images of the tissue being screened. This type of system is known as automated whole breast ultrasound screening. One of the drawbacks of this system is that radiologists are required to learn new techniques for reviewing the ultrasound images, which likewise has resulted in reduced marketplace interest for these systems.

While this automated whole breast ultrasound screening system has been shown in a study to reliably find many small cancers 4-5 mm, which is generally superior to the screening capabilities of handheld ultrasound screening and those of 3D automated whole breast ultrasound screening, its drawbacks have limited its adoption in the marketplace.

### SUMMARY OF THE INVENTION

The present invention is directed to an improved system of ultrasonic screening and diagnostics of cellular tissue. A system controller controls an electromechanical positioning apparatus and an ultrasound apparatus to generate a first set of ultrasound images of cellular tissue. An image analyzer analyzes each generated ultrasound image, and in response to data from the image analyzer, the system controller further controls at least one of the electromechanical positioning apparatus and the ultrasound apparatus. The present invention is also directed to a method for screening and diagnostics of cellular tissue by controlling an electromechanical positioning apparatus and an ultrasound apparatus to generate a first set of ultrasound images of cellular tissue, analyzing the generated ultrasound images, and in response to data from the analysis, further controlling at least one of the electromechanical positioning apparatus and the ultrasound apparatus in response.

In a first separate embodiment of the present invention, a system for screening and diagnostics of cellular tissue includes: an ultrasound apparatus comprising a scan head; an electromechanical positioning apparatus comprising an articulated arm, the scan head coupled to a free end of the articulated arm; a system controller communicably coupled to the ultrasound apparatus and to the electromechanical positioning apparatus, the system controller configured to control the electromechanical positioning apparatus to move the scan head adjacent the cellular tissue while controlling the ultrasound apparatus to generate a first set of ultrasound images of the cellular tissue; and an image analyzer communicably coupled to the ultrasound apparatus to receive ultrasound images and to the system controller, wherein in response to receiving each ultrasound image of the first set of ultrasound images, the image analyzer is configured to analyze each received ultrasound image to identify a potential abnormality in and a location of the potential abnormality within the cellular tissue, and following analysis of the first set of ultrasound images, to communicate potential abnormality data to the system controller, the potential abnormality data comprising one of the location of the potential abnormality within the cellular tissue or an indicator that no abnormality is identifiable within the first set of ultrasound images; wherein, in response to receiving the potential abnormality data including the location of the potential abnormality, the system controller is configured to control the electromechanical positioning apparatus to move the scan head to a first position adjacent the location of the potential abnormality within the cellular tissue, and starting from the first position, to control at least one of the electromechanical positioning apparatus and the ultrasound apparatus to generate a second set of ultrasound images of the potential abnormality, the second set of ultrasound images and the first set of ultrasound images each detailing different aspects of the potential abnormality.

In a second separate embodiment of the present invention, a system for screening and diagnostics of cellular tissue includes: an ultrasound apparatus comprising a scan head; an electromechanical positioning apparatus comprising an articulated arm and an optical sensor, the scan head and the optical sensor coupled to a free end of the articulated arm; a system controller communicably coupled to the ultrasound apparatus and to the electromechanical positioning apparatus, the system controller being configured to control the electromechanical positioning apparatus, using optical data received from the optical sensor, to move the scan head adjacent the cellular tissue while controlling the ultrasound apparatus to generate a first set of ultrasound images of the cellular tissue; and an image analyzer communicably coupled to the ultrasound apparatus to receive ultrasound images and to the system controller, wherein in response to receiving each ultrasound image of the first set of ultrasound images, the image analyzer is configured to analyze each received ultrasound image to determine whether each ultrasound image conforms with predetermined parameters, to generate image optimization data indicating whether each ultrasound image conforms with predetermined parameters, and to communicate the image optimization data to the system controller; wherein in response to receiving the image optimization data indicating that one of the ultrasound images does not conform to the predetermined parameters, the system controller is configured to perform at least one of: control the electromechanical positioning apparatus to adjust a position of the scan head with respect to the cellular tissue and control the ultrasound apparatus to adjust ultrasound image acquisition parameters.

In a third separate embodiment of the present invention, a method for screening and diagnostics of cellular tissue includes: controlling, using a system controller, an electromechanical positioning apparatus and an ultrasound apparatus to move an articulated arm of the electromechanical positioning apparatus and to move a scan head of the ultrasound apparatus adjacent the cellular tissue, the scan head coupled to a free end of the articulated arm; controlling, using the system controller while moving the scan head adjacent the cellular tissue, the ultrasound apparatus to generate a first set of ultrasound images of the cellular tissue; analyzing, using an image analyzer, each ultrasound image of the first set of ultrasound images to identify a potential abnormality in the cellular tissue and to determine a location of the potential abnormality within the cellular tissue, and generating potential abnormality data comprising one of the location of the potential abnormality within the cellular tissue or an indicator that no abnormality is identifiable within the first set of ultrasound images; receiving, at the system controller, the potential abnormality data; and controlling, using the system controller and in response to receiving the potential abnormality data including the location of the potential abnormality, the electromechanical positioning apparatus to move the scan head to a first position adjacent the location of the potential abnormality within the cellular tissue; and controlling, using the system controller and starting from the first position, at least one of the electromechanical positioning apparatus and the ultrasound apparatus to generate a second set of ultrasound images of the potential abnormality, the second set of ultrasound images and the first set of ultrasound images detailing different aspects of the potential abnormality.

In a fourth separate embodiment of the present invention, a method for screening and diagnostics of cellular tissue includes: controlling, using a system controller, an electromechanical positioning apparatus and an ultrasound apparatus to move an articulated arm of the electromechanical positioning apparatus and to move a scan head of the ultrasound apparatus adjacent the cellular tissue, the scan head coupled to a free end of the articulated arm; controlling, using the system controller while moving the scan head adjacent the cellular tissue, the ultrasound apparatus to generate a first set of ultrasound images of the cellular tissue; analyzing, using an image analyzer, each ultrasound image of the first set of ultrasound images to determine whether each ultrasound image is conforming with predetermined parameters; generating, using the image analyzer, image optimization data indicating whether each ultrasound image is conforming with the predetermined parameters; communicating, using the image analyzer, the image optimization data to the system controller; and controlling, using the system controller in response to receiving the image optimization data indicating that at least one of the ultrasound images does not conform with the predetermined parameters, at least one of: the electromechanical positioning apparatus, to adjust a position of the scan head with respect to the cellular tissue, and the ultrasound apparatus to adjust ultrasound image acquisition parameters.

Accordingly, an improved system and method for screening and diagnostics of cellular tissue are disclosed. Advantages of the improvements will be apparent from the drawings and the detailed description provided herein.

### BRIEF DESCRIPTION OF THE DRAWINGS

FIG. 1 schematically illustrates a system for screening and diagnostics of cellular tissue;

FIG. 2 illustrates a robotic arm with a scan head affixed thereto;

FIG. 3 illustrates a partial view of the scan head coupled to a scan head mount;

FIG. 4 is a flow diagram detailing a first screening and diagnostics process;

FIG. 5 illustrates a camisole fabric covering worn on a body to facilitate screening and diagnostics;

FIG. 6 illustrates a plan view of visual markers placed on cellular tissue to facilitate the screening and diagnostics process;

FIG. 7 schematically illustrates the generation of ultrasound images in a plurality of scan rows using the visual markers;

FIG. 8a illustrates a scan head coupled to a scan head mount and positioned over cellular tissue;

FIG. 8b illustrates a scan head and a scan head mount positioned to generate ultrasound images from cellular tissue;

FIG. 8c illustrates a scan head and a scan head mount positioned to generate ultrasound images of a potential abnormality with cellular tissue; and

FIG. 9 is a flow diagram detailing a second screening and diagnostics process.

### DETAILED DESCRIPTION

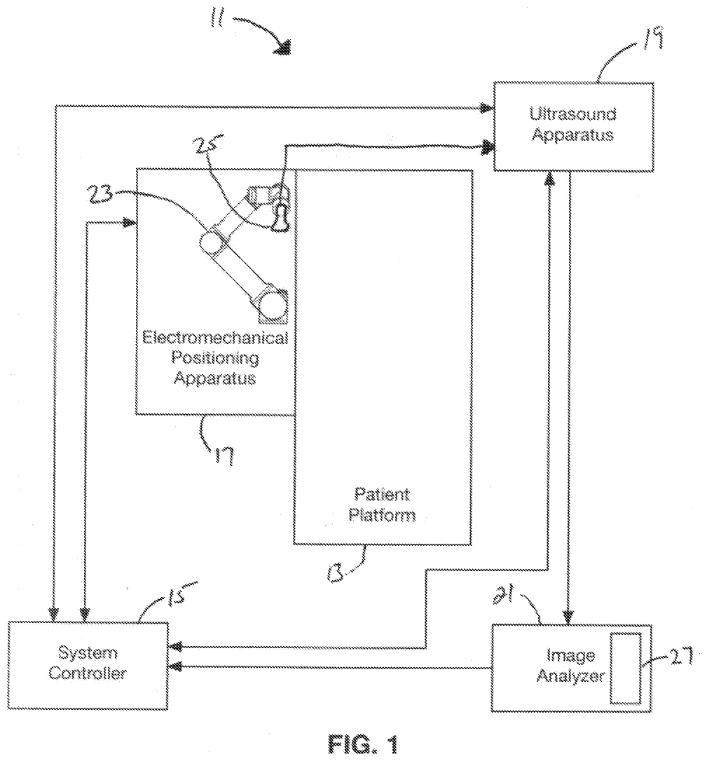

Turning in detail to the drawings, FIG. 1 shows a system 11 for screening and diagnostics of cellular tissue. The system 11 is situated about a patient platform 13, which is provided to steady the patient during the screening and diagnostics process. In certain embodiments, the examination table 13 may be incorporated into and provide a base for components of the system 11. The system 11 includes a system controller 15 that is communicably coupled to an electromechanical positioning apparatus 17, an ultrasound apparatus 19, and an image analyzer 21. The electromechanical positioning apparatus 17 includes an articulated arm 23, and the ultrasound apparatus 19 includes a scan head 25 coupled to a free end of the articulated arm 23. The articulated arm 23, as described in greater detail below, may be a robotic arm which enables the free end to move with six independent degrees of freedom. The system 11 may be assembled using commercially available components for both the electromechanical positioning apparatus 17 and the ultrasound apparatus 19. It is anticipated that the functionality of such commercially available components are sufficient to perform the processes herein. However, minor external modifications may be required to such components in order to physically integrate the components into the system 11.

The system controller 15 may be any appropriate and commercially available computer or other programmable device which is capable of being programmed for controlling both the electromechanical positioning apparatus 17 and the ultrasound apparatus 19 to perform the screening and diagnostics processes described in detail below. Specifically, the system controller 15 is programmed to send control signals to each of the electromechanical positioning apparatus 17 and the ultrasound apparatus 19 to begin and during the screening and diagnostics process. Control signals to the electromechanical positioning apparatus 17 serve to move the scan head 25 to a desired position, to move the scan head 25 at a desired speed, and/or to move the scan head 25 along a desired path. Control signals to the ultrasound apparatus 19 serve to control the timing of when ultrasound images are generated and to control certain aspects of the ultrasound images, such as the image depth and/or the image mode with which the ultrasound apparatus 19 uses to generate the ultrasound images.

The image analyzer 21 is also communicably coupled to the ultrasound apparatus 19 in order to receive ultrasound images which are to be analyzed during the screening and/or diagnostic processes. The image analyzer 21 may be any appropriate and commercially available computer or other programmable device which is capable of being programmed for performing image analysis as described herein. In embodiments in which the system controller 15 is a personal computer or similar computing device, the image analyzer 21 may be a subsystem within the system controller 15. In such embodiments the image analyzer 21 may be a software subsystem, a hardware subsystem, or a combination of both.

In certain embodiments, a machine learning module 27 is included as part of the image analyzer 21, and the machine learning module 27 is a software subsystem which uses artificial intelligence for analyzing the ultrasound images. The machine learning module 27 is trained using ultrasound images from past screenings of similar tissues so that it can quickly determine, in real-time, whether any of the ultrasound images depict potential abnormalities within the cellular tissue. The machine learning module 27 may also be trained to determine, in real-time, whether the scan head 25 is positioned as desired with respect to the cellular tissue, and whether the ultrasound images generated by the ultrasound apparatus 19 conform to desired parameters.

The machine learning module 27 is, in certain embodiments, may be an artificial intelligence technology referred to as a deep convolutional neural networks (CNN). CNN is a well-known type of artificial intelligence, and as such is not discussed here in detail. CNN is well-suited for analyzing images, such as ultrasound images, after it has been trained using sample images. It is known that increasing the number of sample images used to train a CNN results in increased accuracy for the subsequent image analysis. Importantly, CNN have been developed and studied by different scientific groups globally and have proven their efficiency and excellent sensitivity in detecting abnormalities within ultrasound images of cellular tissue, including breast tissue.

In embodiments in which the system controller 15 is a programmable computer which includes sufficient digital storage space (such as RAM, a hard drive, etc.), all the ultrasound images and all the data generated by the image analyzer 21 may be stored within digital storage space before and after the screening and diagnostics process. Alternatively a separate digital storage module may be included as part of the system 11 and communicably coupled to the other components so that the ultrasound images and data may be stored within the separate digital storage module. Still in other embodiments, the system controller 15 may be communicably coupled to a wide area network so that the ultrasound images and data may be stored may be communicated to a remote storage module. Such a remote storage module may be located anywhere, with the data being transmitted thereto over the Internet. In still other embodiments, a combination of local and remote storage may be used.

Details about the operation of each of the components of the system 11 is described in greater detail below.

One or both of the system controller 15 and the ultrasound apparatus 19 may include a display screen so that ultrasound images and other data generated during the screening and diagnostics process may be displayed to the person operating the system 11 and/or to the patient and/or to a radiologist.

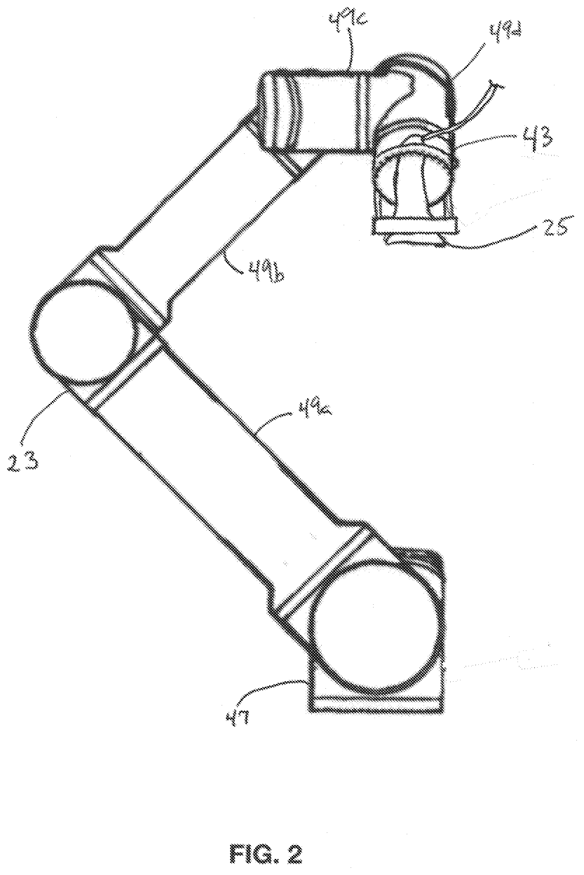

FIG. 2 shows the articulated arm as a robotic arm 41 having a scan head mount 43 at the free end 45 thereof. The scan head 25 is secured to the scan head mount 43. The mechanical structure of the scan head mount 43 serves two important tasks, first as the last arm segment of the robotic arm 41, and second as a secure attachment point for the scan head 25 to the free end 45 of the robotic arm 41. Thus, due to the varying shapes and sizes of scan heads used with ultrasound systems that are commercially available, the structural design of the scan head mount 43 may widely vary.

The articulated arm 23 includes a rotatable base 47 and a plurality of arm segments 49a-d, and each arm segment 49a-d is rotationally coupled to each adjoining arm segment 49a-d, the first arm segment 49a is rotationally coupled to the rotatable base 47, and the fourth arm segment 49d is rotationally coupled to the scan head mount 43. Such robotic arms are well-known in the industry to provide movement for the free end 45 in six independent degrees of freedom. One example of such a robotic arm that is commercially available is the UR5e model manufactured by Universal Robots A/S from Denmark, Energivej 25 DK-5260 Odense S.

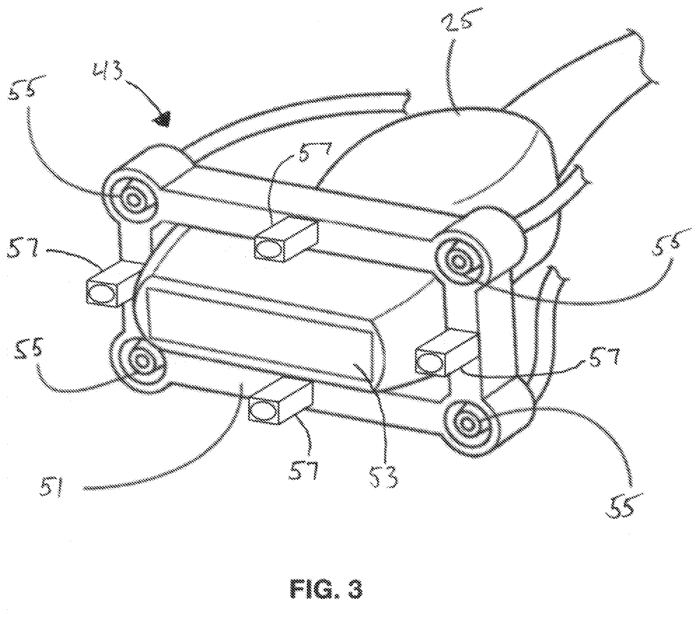

A partial view of an exemplary scan head mount 43, with the scan head 25 coupled thereto, is shown in FIG. 3. In this embodiment, the scan head mount 43 includes a frame 51 that is positioned near the transducer 53 of the scan head 25. As shown, the frame 51 has a rectangular shape, however, in practice the frame 51 may have any shape desirable. Four optical sensors 55 are included at the corners of the frame 51, and four pressure sensors 57 are included along the sides of the frame 51. When the transducer 53 is facing the cellular tissue, the optical sensors 55 and the pressure sensors 57 are also facing the cellular tissue. The optical sensors 55 may generate optical data at all times when the system 11 is active. In comparison, the pressure sensors 57 only generate pressure data then the transducer is placed against the cellular tissue and one or more of the pressure sensors 57 also comes into contact with the cellular tissue or, when a camisole is present and covering the cellular tissue as described below, with the fabric of the camisole.

Both the optical sensors 55 and the pressure sensors 59 provide feedback to the system controller 15 for purposes of controlling the robotic arm 41 and thus also controlling the position of the scan head 25 with respect to the cellular tissue during the screening and diagnostics process. In particular, optical data provided by the optical sensors 55 aid the system controller 15 in controlling the distance between the scan head 25 and the cellular tissue. The optical data from the optical sensors 55 also indicate to the system controller 13 where to start and stop the screening process and how to control the movement of the scan head 25 during the screening process. In certain embodiments, the optical data from the optical sensors 55 may also be used by the system controller 15 to determine the distance between the transducer and the cellular tissue. Pressure data provided by the pressure sensors 57 aid the system controller 15 in controlling the amount of pressure the scan head 25 applies against the cellular tissue, and the pressure data also aids in controlling the distribution of the applied pressure across the full length and width of the transducer 53. In controlling the pressure of the scan head 25 against the cellular tissue, the system controller 15 is able to aid the ultrasound apparatus in generating quality ultrasound images, and the pressure can be controlled to help maintain the comfort of the patient.

The optical sensors 55 may operate in any wavelength that is appropriate for a patient examination setting. For example, they optical sensors 55 may operate in the range of visual light, or they may operate within the infrared spectra. In certain embodiments, it may be desirable to also include a light source to illuminate the patient and the cellular tissue during the screening and diagnostics process. Such a light source may be an ambient light source which provides broad illumination, or alternatively the light source may be mounted to the frame 51. The inclusion of a light source may also serve to illuminate the visual markers placed on or adjacent the cellular tissue to guide the screening and diagnostics process.

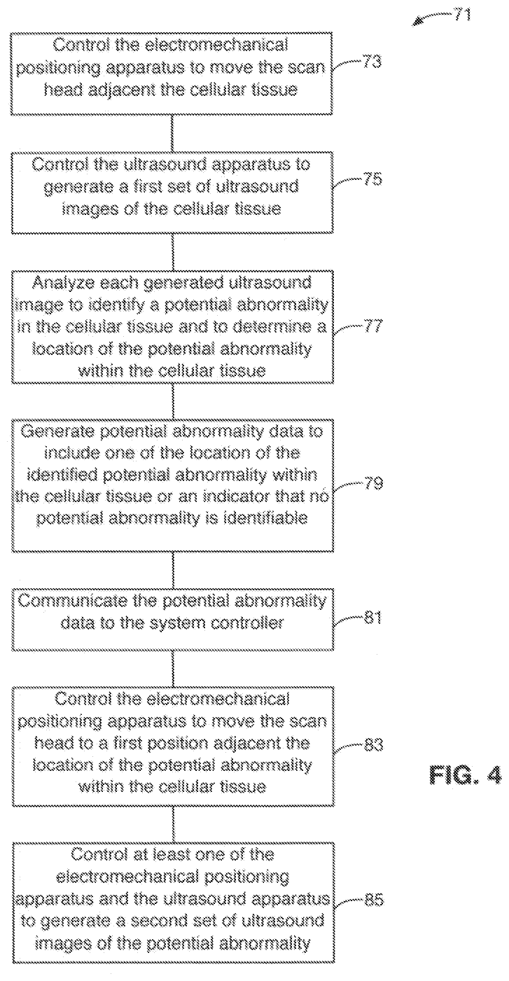

A first embodiment of the process for screening and diagnostics of cellular tissue is shown in the flow chart 71 of FIG. 4. While this process is discussed in the context of screening and diagnostics of breast tissue, it should be recognized that the process could be easily adapted for the screening and diagnostics of any in situ cellular tissue. The process depicted in this flow chart 71 may be programmatically implemented using the components of the system 11. The start of the process is to control 73 the electromechanical positioning apparatus to move the scan head adjacent the cellular tissue. In moving the scan head, the articulated arm of the electromechanical positioning apparatus is controlled to move the scan head in a methodical manner along the entirety of the cellular tissue.

Before the process begins, the patient is asked to lie on the examination table in the supine position with their arm positioned over their head. A pillow may be placed under the patient's back to help flatten the breast in a comfortable position for the screening and diagnostics process.

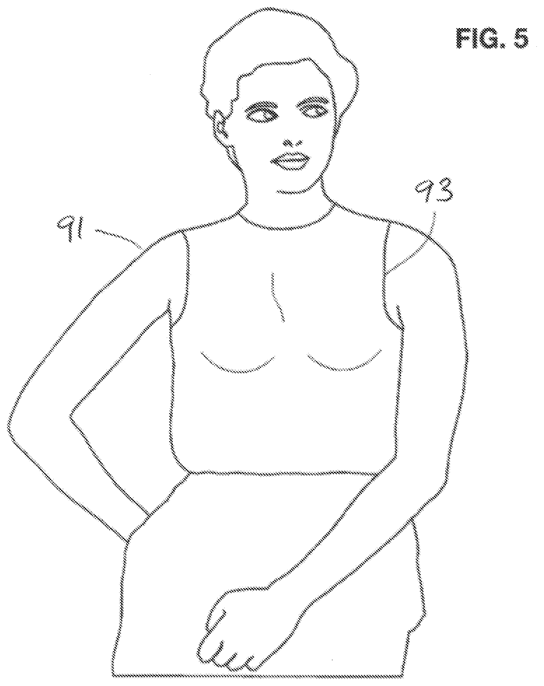

As preparation for moving the scan had along the cellular tissue, in cases when the cellular tissue is breast tissue, it is beneficial for the patient 91 to be wearing a camisole 93 as shown in FIG. 5. The camisole 93 is a bra-like covering that aids in holding the breast tissue in position during the screening and diagnostics process, as well as assisting in uniform integrity of ultrasound image gathering by reducing information loss from ultrasonic shadowing. In addition, during the screening process, a medical nipple-pad may be placed over the patient's nipple to enhance patient comfort and improve the image quality of the nipple region as the scan head is moved over the nipple. The camisole 93 also provides some modesty for the patient. Current ultrasound technology requires the use of sonographic coupling agent, usually a gel, to exclude any air between the probe and the skin. Therefore, the camisole 93 is formed from a fabric, or combination of fabrics, that is capable of absorbing the gel. Once gel is absorbed, the camisole 93 becomes relatively transparent to ultrasonic energy. The camisole 93 may also be pre-impregnated with the coupling agent, or the agent may be applied by the operator just prior to the screening and diagnostics process, or both. To avoid having the patient pull a gel-soaked covering over their head after the screening and diagnostics process is completed, the camisole 93 may be designed to dismantle after use. In an exemplary design, the back (not shown) of the camisole 93 may include a seam that is constructed with chain stitching that is easily removed, thereby enabling the patient to slip the camisole 93 off over their arms.

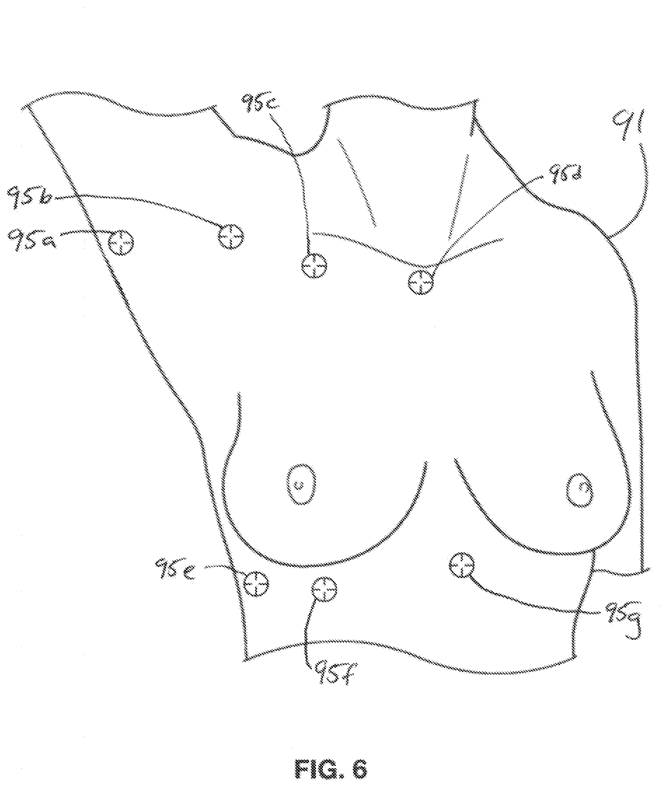

Additional preparation for moving the scan head is by visual markers 93 being placed on or adjacent the cellular tissue (breast tissue here) as shown in FIG. 6. Here, the visual markers 95a-g are shown placed directly on the skin of the patient 91, but in other embodiments the visual markers may be place directly on the camisole 93. The visual markers 95a-g serve to mark anatomical features of the patient so that the entirety of the breast tissue can be included as part of the screening and diagnostics process. The anatomical features, and thus the relative positional placement of the visual markers 95a-g, are entirely at the discretion of the technician or radiologist operating the system 11. In FIG. 6, visual markers 95a-b mark the upper aspect of the axilla, visual marker 95c is placed at a point within the lower contour of the patient's clavicle, visual marker 95d is placed approximately over the patient's xiphoid process and visual markers 95e-g mark the lower limits for the scanning process and are placed at about the lower limits of the patient's rib cage for purposes of defining the scope of the screening process so that a substantially complete screening of the entire breast tissue is performed. The movement of the scan head along the breast tissue is discussed further below in connection with the generation of ultrasound images.

Turning back to FIG. 4, while the scan head is being moved along the breast tissue, the next step is to control 75 the ultrasound apparatus in order to generate a first set of ultrasound images of the cellular tissue. During this step, the ultrasound apparatus is controlled to generate the first set of ultrasound images of the cellular tissue. During these first two steps 73, 75, electromechanical positioning apparatus and the ultrasound apparatus are operated simultaneously to move the scan head along the cellular tissue and to generate ultrasound images during the scan head movement. By this simultaneous operation, the first set of ultrasound images can be generated to provide a substantially complete screening of the entire breast tissue. The ultrasound images that are generated for the screening process are transverse to the coronal plane cross-sectional images of the cellular tissue of the axilla and breast regions (which are collectively referred to herein as “breast tissue”).

As the system controller 15 controls the electromechanical positioning apparatus 17 to move the scan head 25 while also controlling the ultrasound apparatus 19 to generate ultrasound images, the system controller 15 maintains a correlation between the position of the scan head and the generated ultrasound images. This correlation may take the form of assigning unique numbers to make the correlation, applying a time stamp so that the position and the ultrasound image can later be correlated, or by any other correlation method or technique. By maintaining this correlation, the system controller 15 is able to quickly and precisely identify the position of the scan head 25 at the time a particular ultrasound image was generated, and this information may be used during the diagnostics process.

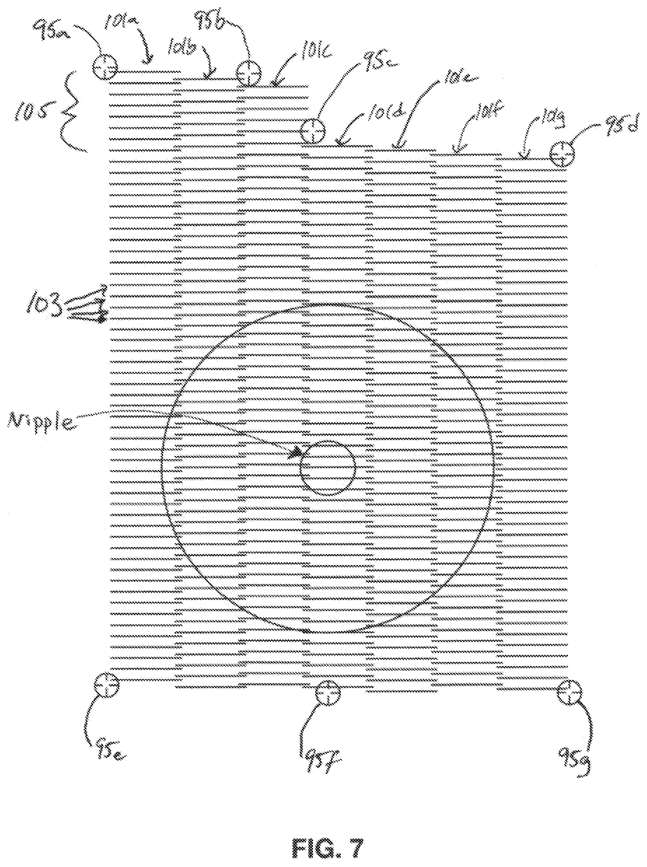

FIG. 7 illustrates the screening of breast tissue by moving the scan head using the electromechanical positioning apparatus 17 and generating the first set of ultrasound images. The electromechanical positioning apparatus 17 is controlled to move the scan head 25 along a plurality of scan rows 101a-g, and within each scan row, the ultrasound apparatus 19 is controlled to generate a series of ultrasound images using the scan head 25. The combination of all the generated ultrasound images in all of the scan rows forms the first set of ultrasound images.

Before actually performing the scanning, the optical sensors 55 are used to identify the position of the visual markers 95a-g by positioning the scan head above the patient with visual markers 95a-g within the field of view of the optical sensors. The optical sensors 55 generate optical data concerning the relative positions of the visual markers 95a-g and communicate the optical data to the system controller. In certain embodiments, it may be desirable to use the optical sensors 55 to create an image of the position of the markers for later reference or future recreation of a screening and diagnostics process. The system controller 15 may then control the electromechanical positioning apparatus to move the scan head to the visual marker 95a. In certain embodiments, the visual marker 95a may have a coloration or marking which identifies it as the starting point for the screening process. In still other embodiments, the visual marker 95a and at least one other of the visual markers 95b-g may be uniquely identified by coloration or markings. The system controller 15, having the visual marker 95a at the starting point identified, may begin the screening process once the scan head is moved to the visual marker 95a. Moreover, by having data concerning the location of each visual marker 95a-g, the control system may substantially autonomously, if not entirely, control the electromechanical positioning apparatus during the screening process.

Alternatively, the position of each of the visual markers 95a-g may be manually provided to the electromechanical positioning apparatus by manual calibration. In such embodiments, the technician would move the scan head to each visual marker 95a-g one at a time so that the position of each visual marker 95a-g may be recorded by the control system. The recorded position would be the rotations settings of all rotational joints of the robotic arm when the scan head is located at one of the visual markers 95a-g.

The starting point of the screening process is at the visual marker 95a, and in first scan row 101a, the series of ultrasound images 103 are generated until the scan head gets to the visual marker 95e, which indicates the end of the scan row 101a. Next, the scan head is returned to a point between visual markers 95a-b to start the second scan row 101b, and movement of the scan head proceeds until it reaches a point between visual markers 95e-f. The ultrasound images 103 in this second scan row 101b have a small amount of overlap with the ultrasound images 103 in the first scan row 101a. In certain embodiments, the overlap may be about 4 mm to 5 mm, however more or less overlap may be used depending upon the quality of the ultrasound images being generated. An overlap such as this is created with all adjacent scan rows to help ensure the first set of ultrasound images provide a substantially complete screening of the entire breast tissue.

Within each scan row, the electromechanical positioning apparatus and the ultrasound apparatus are controlled in order to control, respectively, the speed of movement of the scan head and the rate of generating ultrasound images. By control of these two components in this manner, the screening process can controlled so that spacing between adjacent ultrasound images in a scan row is between about 0.1 mm to 0.4 mm. Such spacing between ultrasound images improves detection of small potential abnormalities and increases the resolution and detail of any potential abnormalities identified by the screening process.

The screening process continues in this same manner for each additional scan row 101c-g, until the visual marker 95g is reached at the end of scan row 101g. With this screening process, using the visual markers 95a-g and the multiple overlapping scan rows 101a-g, the generated ultrasound images provide a substantially complete screening of the entire breast tissue, including tissue in the axilla area 107.

The seven scan rows 101a-g shown are exemplary only, as the number of scan rows will typically depend upon the width of the transducer incorporated into the scan head and the area of tissue marked off by the visual markers 95a-g. In general, visual marker 95a indicates the starting position for the scan head as the screening process begins, visual markers 95a-d indicate the starting point for each the scan rows, visual markers 95e-g indicate the ending point for each of the scan rows, and visual marker 95g indicates the ending position for the scan head at the end of the screening process.

During the screening process, the pressure data is used to maintain as much of the transducer as possible in contact with the breast tissue while the scan head 25 is being moved and ultrasound images are being generated. To achieve this, the pressure data is used to dynamically adjust the angular position of the scan head 25 during the screening process. In addition, the system may programmed to have a threshold pressure set by the technician. This threshold pressure may be set to ensure that the pressure of the scan head on the patient does not exceed this threshold pressure, thereby aiding in maintaining a comfort level for the patient during the screening and diagnostics process.

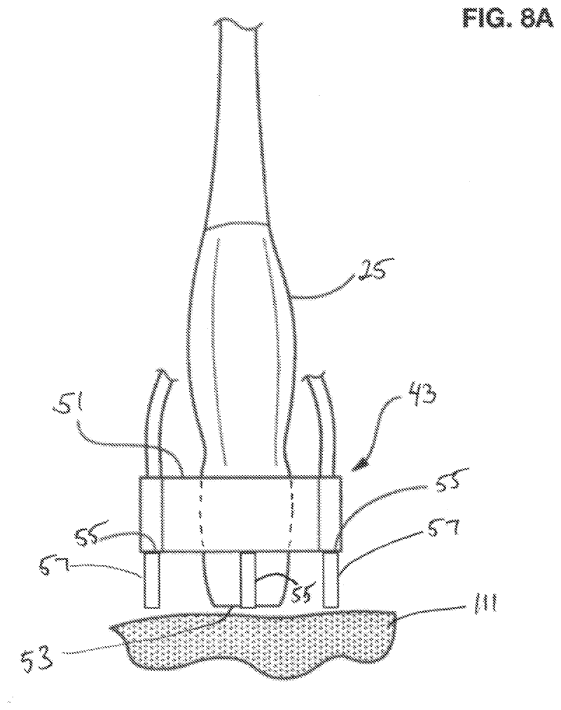

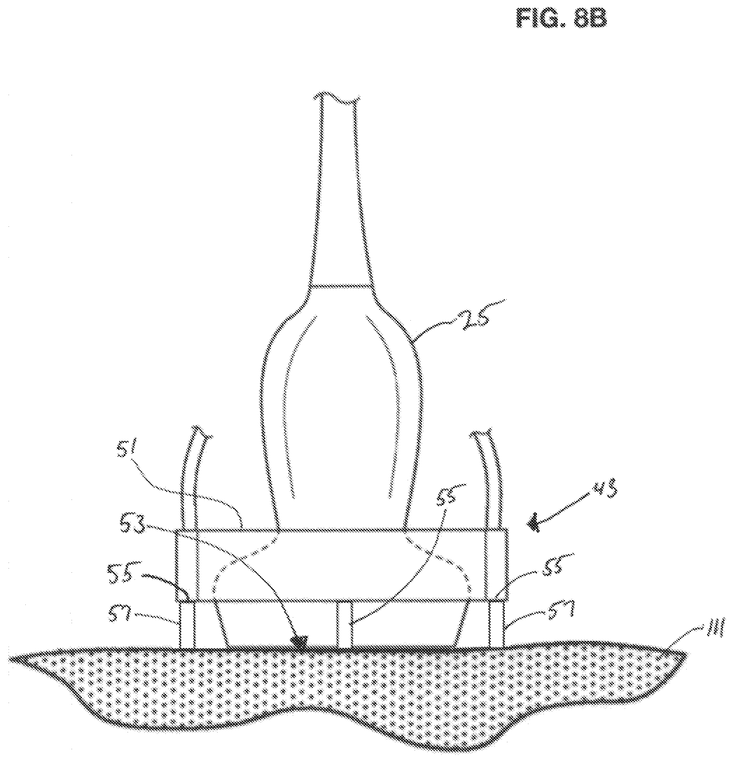

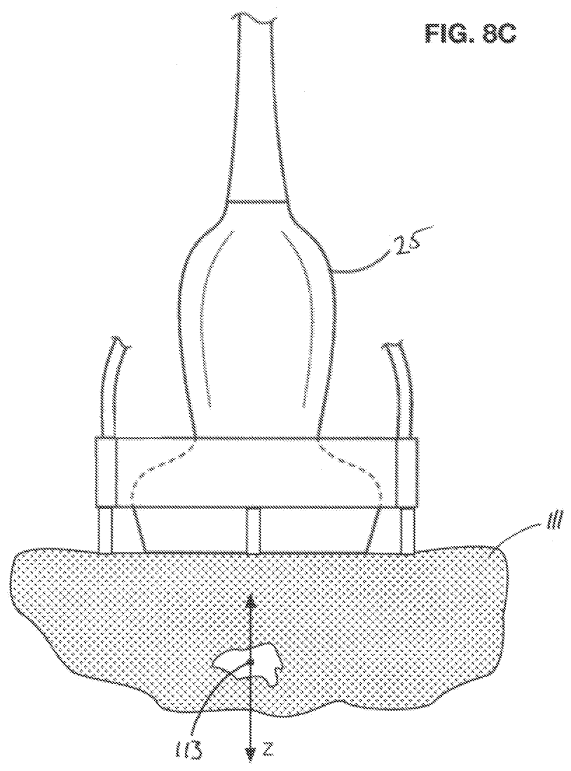

By way of example, FIGS. 8A-B show the scan head in different positions with respect to the cellular tissue. In FIG. 8A, the scan head 25 is shown disposed within the frame 51 of the scan head mount 43 (only partially shown), and the transducer 53 is not in contact with the cellular tissue 111. In this condition of no contact between the transducer 53 and the cellular tissue 111, the optical sensors 55 would be generating optical data and providing that optical data to the system controller 15. The optical data may enable the system controller to determine the distance between the transducer 53 and the cellular tissue 111. In the event that one of the visual markers 95a-g is present within the field of view of the optical sensors, the optical data will indicate this presence. Also in the this condition of no contact between the transducer 53 and the cellular tissue 111, the system controller 15 would be informed that there is no contact based on the pressure data reflecting that none of the pressure sensors 57 are registering a physical pressure. In certain embodiments, this would result in the pressure data having a null value.

FIG. 8B again shows the scan head 25 disposed within the frame 51 of the scan head mount 43 (only partially shown), and the transducer 53 is in contact with the cellular tissue 111. In this condition of contact between the transducer and the cellular tissue 111, both the optical sensors 55 and the pressure sensors 57 would be generating, respectively, optical data and pressure data and providing that data to the system controller 15. Moreover, in embodiments in which the system controller may determine the distance between the transducer 53 and the cellular tissue 111 using the optical data, both the optical data and the pressure data would indicate that the transducer 53 is in full contact with the cellular tissue 111 with the pressure exerted by the transducer 53 on the cellular tissue being approximately evenly distributed. When the pressure is unevenly distributed, pressure data from each of the pressure sensors 55 will be sufficiently different to identify that the pressure is unevenly distributed. In such circumstances, the system controller 15 may also use the pressure data to determine adjustments that should be made in order to better distribute the pressure across the full length and width of the transducer 53.

Returning again to FIG. 4, the next step in the screening and diagnostics process is to analyze 77, using the image analyzer 21, each generated ultrasound image to identify a potential abnormality in the cellular tissue and to determine a location of the potential abnormality within the cellular tissue. As indicated above, this may be performed by the machine learning module 27 that is included as part of the image analyzer 21. Using a CNN with appropriate initial training, this step 77 may be performed in real time as the ultrasound images are being collected and communicated to the image analyzer 21. As part of analyzing each ultrasound image, because potential abnormalities generally are of a size that they may create image artifacts on multiple adjacent ultrasound images, it is also helpful to analyze each ultrasound image in conjunction with one or more sequentially prior generated ultrasound images and/or one or more sequentially subsequent generated ultrasound images. Analyzing the ultrasound images in this manner can increase the success of identifying a potential abnormality, as opposed to analyzing each ultrasound image individually.

Following conclusion of the screening and diagnostics process and confirmation by a radiologist that a potential abnormality does or doesn't exist within the cellular tissue, the ultrasound images from the just completed screening and diagnostics process may be used to further train the CNN. In this manner, the capabilities of the machine learning module 27 for screening cellular tissue will continue to improve following every process.

When the machine learning module 27 identifies a potential abnormality with the cellular tissue, that potential abnormality will appear in multiple adjacent ultrasound images within a single scan row or across two adjacent scan rows. From amongst the ultrasound images in which the potential abnormality appears, the image analyzer 21 will assign one of the ultrasound images in the approximate center of the group of ultrasound images to be the representative ultrasound image for that potential abnormality. The image analyzer 21 will also identify an approximate planar center of the potential abnormality within the plane of the representative ultrasound image. The representative ultrasound image together with the planar center of the potential abnormality are used by the image analyzer 21 to identify the location of the potential abnormality within the cellular tissue.

Once the potential abnormality is identified, the next step is to generate 79, using the image analyzer 21, potential abnormality data. This step is performed once the entire first set of ultrasound images has been generated by the ultrasound apparatus 19 and analyzed by the image analyzer 21. When no potential abnormality has been identified by the image analyzer 21, the potential abnormality data includes an indicator that no potential abnormality is identifiable within the cellular tissue. In instances when a potential abnormality is identified, the potential abnormality data includes identification of all ultrasound images which show part of the potential abnormality, identification of the reference ultrasound image, and identification of the planar center of the potential abnormality within the reference ultrasound image. Once the potential abnormality data is generated, the image analyzer 21 communicates 81 the potential abnormality data to the system controller 15.

Upon receiving the potential abnormality data that indicates the presence of a potential abnormality within the cellular tissue, the system controller 15 controls 83 the electromechanical positioning apparatus 17 to move the scan head 25 to a first position that is adjacent the location of the potential abnormality identified within the cellular tissue. This first position is the location of the scan head 25 when the reference ultrasound image was generated during the control step 75. In FIG. 8C, the scan head 25 is shown in the first position adjacent the cellular tissue 111. As determined by the image analyzer 21, the planar center 113 of the potential abnormality is also marked. In addition, a reference z-axis is identified in order to facilitate the generation of additional sets of ultrasound images of the potential abnormality. The system controller 15 may use the representative ultrasound image and the planar center to define the reference z-axis for the potential abnormality, and the scan head 25 may be approximately centered over this reference z-axis. This reference z-axis passes through the center of the potential abnormality within the cellular tissue and is defined by the position and orientation of the reference ultrasound image at the time it was generated. Specifically, the reference z-axis passes through the planar center of the potential abnormality within the reference ultrasound image, lie in the plane of the representative ultrasound image, and be approximately normal to the position of the transducer when the reference ultrasound image was generated.

Once the scan head 25 is moved to the first position, then the system controller 15 controls 85 at least one of the electromechanical positioning apparatus 17 and the ultrasound apparatus 19 to generate a second set of ultrasound images of the potential abnormality. The manner in which this second set of ultrasound images is generated will detail different aspects of the potential abnormality as compared to the first set of ultrasound images. This second set of ultrasound images will provide diagnostic information concerning the potential abnormality, such that when a radiologist considers the second set of ultrasound images, either on its own or in combination with the first set of ultrasound images, the radiologist is able to make a diagnostic evaluation for further treatment of the potential abnormality. In some processes, a third set of ultrasound images of the potential abnormality may be generated, with the third set of ultrasound images detailing different aspects of the potential abnormality as compared to both the first set of ultrasound images and the second set of ultrasound images.

The second, third, and any additional sets of ultrasound images are intended for diagnostics purposes and may be generated by one or more of the following:
1. The electromechanical apparatus 17 may be controlled to rotate the scan head about the reference z-axis in order to generate orthogonal views of the potential abnormality.
2. The ultrasound apparatus 19 may be controlled in a real-time shear wave elastography mode to generate an image set of ultrasound images showing the potential abnormality and some of the surrounding tissues in order to improve the specificity of automated conclusions that are appropriate. Shear wave elastography is a mode that is found in some commercially available ultrasound systems. If the ultrasound system supports 3D shear wave elastography, then mode may also be used to generate an additional set of ultrasound images.
3. The ultrasound apparatus 19 may be controlled in a microflow or microvascular flow analysis mode. This is also a mode that is common to most commercially available ultrasound systems. This mode provides microvasculature low-velocity color Doppler mapping of the potential abnormality.
4. The ultrasound apparatus 19 may be controlled in a mode with compounding imaging turned off. This is also a mode that is common to most commercially available ultrasound systems, and it can help reduce the impact of shadowing effects.
5. The ultrasound apparatus 19 may be controlled in a mode with harmonic imaging turned on. This is also a mode that is common to most commercially available ultrasound systems. Ultrasound images generated using this mode may be stored as a 2D image with harmonics data.

If more than one potential abnormality is identified within the first set of ultrasound images, then steps 83 and 85 above are repeated for each potential abnormality.

Once the screening and diagnostics process is completed for the breast tissue on one side of a patient's body, then the entire process is repeated for the breast tissue on the other side of the patient's body. In this way, a full scan and full diagnostics are generated during the same visit to the radiologist without any return visits being required before diagnosis. This is in contrast to the current process with mammograms and follow-up appointments for diagnosis, a process that can be both frustrating an anxiety provoking.

As part of the screening and diagnostics process described above, all the data generated by the system 11 during the screening and diagnostics may be saved in a raw format for later review and/or processing. In addition, the end results of processed data may also be saved with the raw data. For example, such end results may include short cine loops created for purposes of presentation of the diagnostics information to the radiologist, it might include a calculation of the volume of the potential abnormality, or it might include measurements of the location of the potential abnormality with respect to other physical landmarks such as the nipple, the skin, and the chest wall. The system 11 may include adequate memory or other storage space for use as short term storage of data. However, for long term storage, it may be more convenient to have the system controller 15 communicably coupled to a wide area network so that all data from a screening and diagnostics for a patient may be stored off-site for later access as needed.

By performing the screening and diagnostics process simultaneously, valuable time for all involved, including the patient and the technician and/or radiologist, is conserved. This process takes less time than the traditional mammogram screening with a follow-up handheld ultrasound diagnostics, and it provides greater ability to detect small abnormalities along with all the diagnostic information from the same patient visit. In addition, the ultrasound images and diagnostics information may be presented in a format that is already familiar to radiologists, meaning that there is little to no learning curve to read the materials generated by the screening and diagnostics system and process disclosed herein.

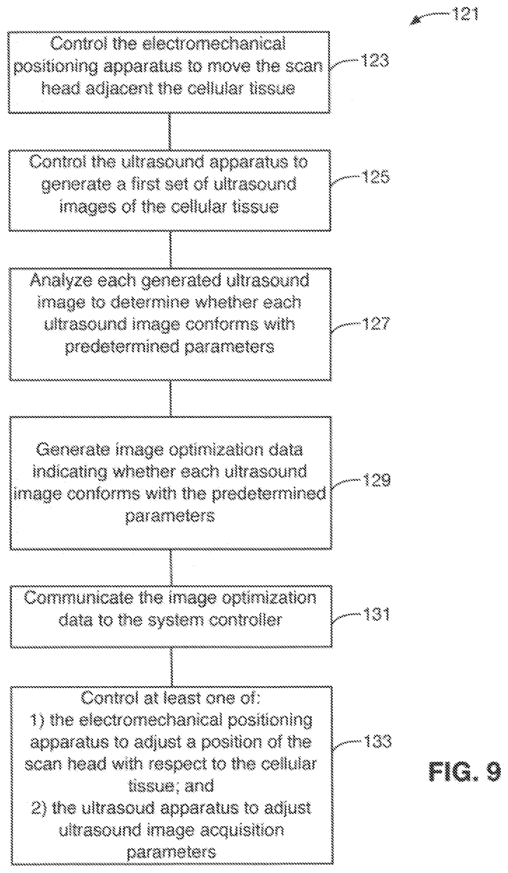

A second embodiment of a process for screening and diagnostics of cellular tissue is shown in the flow chart 121 of FIG. 9. While this process is discussed in the context of screening and diagnostics of breast tissue, it should be recognized that the process could be easily adapted for the screening and diagnostics of any in situ cellular tissue. The process depicted in this flow chart 121 may be programmatically implemented using the components of the system 11. The start of the process is to control 121 the electromechanical positioning apparatus 17 to move the scan head 25 adjacent the cellular tissue. In moving the scan head 25, the articulated arm of the electromechanical positioning apparatus 17 is controlled to move the scan head 25 in a methodical manner along the entirety of the cellular tissue.

As discussed in detail above, before the process begins, the patient is asked to lie on the examination table in the supine position with their arm positioned over their head. A camisole 93 may also be worn by the patient during the procedure, and visual markers may be placed directly on the breast tissue or on the camisole 93 if one is worn.

While the scan head 25 is being moved along the breast tissue, the next step is to control 125 the ultrasound apparatus in order to generate a first set of ultrasound images of the cellular tissue. During this step, the ultrasound apparatus is controlled to generate the first set of ultrasound images of the cellular tissue. During these first two steps 123, 125, electromechanical positioning apparatus and the ultrasound apparatus are operated simultaneously to move the scan head along the cellular tissue and to generate ultrasound images during the scan head movement. By this simultaneous operation, the first set of ultrasound images can be generated to provide a substantially complete screening of the entire breast tissue. The ultrasound images that are generated for the screening process are transverse to the coronal plane cross-sectional images of the cellular tissue of the axilla and breast regions (which are collectively referred to herein as “breast tissue”).

As the system controller 15 controls the electromechanical positioning apparatus 17 to move the scan head 25 while also controlling the ultrasound apparatus 19 to generate ultrasound images, the system controller 15 maintains a correlation between the position of the scan head and the generated ultrasound images as previously discussed. The screening of the breast tissue is performed in the same manner discussed above, by moving the scan head 25 using the electromechanical positioning apparatus 17 along a plurality of scan rows, and within each scan row, the ultrasound apparatus 19 is controlled to generate a series of ultrasound images using the scan head 25. The combination of all the generated ultrasound images in all of the scan rows forms the first set of ultrasound images.

The next step in the screening and diagnostics process is to analyze 127, using the image analyzer 21, each generated ultrasound image to determine whether each ultrasound image conforms with predetermined parameters. As indicated above, this may be performed by the machine learning module 27 that is included as part of the image analyzer 21. Using a CNN with appropriate initial training, this step 127 can effectively be performed in real time as the ultrasound images are being collected and communicated to the image analyzer 21. As part of analyzing each ultrasound image, the image analyzer 21 looks for artifacts within the ultrasound images which indicate that adjustments may be needed to settings of the ultrasound apparatus 19. Such settings may include the depth of the images, B mode gain, TGC adjustments, adjustment of the focal point positioning, adjusting transducer frequencies to account for the thickness of the cellular tissue being imaged, and compensating for acoustic shadowing caused by the patient's skin. In addition, the image analyzer 21 looks for artifacts within the ultrasound images which indicate that adjustments may be needed to the amount of pressure exerted on the breast tissue by the scan head 25. These are all artifacts and subsequent adjustments that a skilled ultrasound technician or radiologist might perform manually, but can instead be performed by the image analyzer 21. The artifacts are therefore well-known and recognized by those of skill in the art.

The next step is to generate 129 optimization data indicating whether each ultrasound image conforms, or does not conform, to the predetermined parameters. In this step 129, when one of the ultrasound images conforms to the predetermined parameters, the optimization data may include a null set. Conversely, when one of the ultrasound images does not conform to the predetermined parameters, the image optimization data includes an indicia of non-conformance and a reference to indicate a classification of the non-conformance. Once the image optimization data is generated, the image analyzer 21 communicates 131 the image optimization data to the system controller 15.

Upon receiving the image optimization data that indicates non-conformance of an ultrasound image, the system controller 15 controls 133 at least one of the electromechanical positioning apparatus 17 and the ultrasound apparatus 19 to correct the non-conformance. The system controller 15 will control the electromechanical positioning apparatus 17 when the classification of the non-conformance indicates that an adjustment in the pressure of the scan head 25 against the cellular tissue is needed. Similarly, the system controller 15 will control the ultrasound apparatus 19 when the classification of the non-conformance indicates that an adjustment to one or more parameters of the ultrasound apparatus 19. These adjustments take place in real-time to the acquisition of subsequent ultrasound images, just as analyzing the ultrasound images for conformance takes place in real time. By making such real-time adjustments to the generation of ultrasound images, the system controller 21 is better able to continue generating high quality ultrasound images throughout generation of ultrasound images for the first set of ultrasound images. Having high quality ultrasound images, of course, can lead directly to high quality screening and diagnostics process.

Accordingly, an improved system and method for screening and diagnostics of cellular tissue are disclosed. Although embodiments and applications of this invention have been shown and described, it would be apparent to those skilled in the art that many more modifications are possible without departing from the inventive concepts herein. The invention, therefore, is not to be restricted except in the spirit of the appended claims.

What is claimed is:

1. A system for screening and diagnostics of cellular tissue, the system comprising:
an ultrasound apparatus comprising a scan head;
an electromechanical positioning apparatus comprising an articulated arm, the scan head coupled to a free end of the articulated arm;
a system controller communicably coupled to the ultrasound apparatus and to the electromechanical positioning apparatus, the system controller configured to control the electromechanical positioning apparatus to move the scan head adjacent the cellular tissue while controlling the ultrasound apparatus to generate a first set of ultrasound images of the cellular tissue; and
an image analyzer communicably coupled to the ultrasound apparatus to receive ultrasound images and to the system controller, wherein in response to receiving each ultrasound image of the first set of ultrasound images, the image analyzer is configured to analyze each received ultrasound image to identify a potential abnormality in and a location of the potential abnormality within the cellular tissue, and following analysis of the first set of ultrasound images, to communicate potential abnormality data to the system controller, the potential abnormality data comprising one of the location of the potential abnormality within the cellular tissue or an indicator that no abnormality is identifiable within the first set of ultrasound images;
wherein, in response to receiving the potential abnormality data including the location of the potential abnormality, the system controller is configured to control the electromechanical positioning apparatus to move the scan head to a first position adjacent the location of the potential abnormality within the cellular tissue, and starting from the first position, to control at least one of the electromechanical positioning apparatus and the ultrasound apparatus to generate a second set of ultrasound images of the potential abnormality, the second set of ultrasound images and the first set of ultrasound images each detailing different aspects of the potential abnormality.

2. The system of claim 1, wherein the articulated arm is configured to move the scan head in six independent degrees of freedom.

3. The system of claim 1, wherein the second set of ultrasound images is generated by at least one of:
controlling the electromechanical positioning apparatus to rotate the scan head about a z-axis passing through the potential abnormality;
controlling the ultrasound apparatus to operate in a shear-wave elastography mode;
controlling the ultrasound apparatus to operate in a Doppler mode;
controlling the ultrasound apparatus to operate in a mode without spatial compounding imaging; and
controlling the ultrasound apparatus to operate in a mode using harmonic imaging.

4. The system of claim 1, wherein following generation of the second set of ultrasound images, the system controller is further configured to control at least one of the electromechanical positioning apparatus and the ultrasound apparatus to generate a third set of ultrasound images of the potential abnormality, the third set of ultrasound images, the second set of ultrasound images, and the first set of ultrasound images each detailing different aspects of the potential abnormality.

5. The system of claim 1, wherein the first set of ultrasound images comprises a plurality of ultrasound images generated across two or more scan rows.

6. The system of claim 1, the image analyzer comprising a machine learning module trained for ultrasound image analysis, wherein the machine learning module is configured to analyze each ultrasound image of the first set of ultrasound images.

7. The system of claim 6, the machine learning module configured to analyze each received ultrasound image in conjunction with at least one of one or more sequentially prior generated ultrasound images and one or more sequentially subsequent generated ultrasound images.

8. The system of claim 1, wherein the electromechanical positioning apparatus further comprises an optical sensor coupled to the free end of the articulated arm, and wherein the system controller is communicably coupled to the optical sensor to receive optical data generated by the optical sensor.

9. The system of claim 8, the first set of ultrasound images being generated across two or more scan rows, wherein the system controller is configured to determine, using the optical data, a first set starting point for the first set of ultrasound images, a scan row starting point for each scan row, a scan row ending point for each scan row, and a first set ending point for the first set of ultrasound images.

10. The system of claim 9, wherein the optical sensor is configured to generate the optical data using a plurality of visual markers disposed on or adjacent the cellular tissue, the plurality of visual markers representing the first set starting point for the first set of ultrasound images, the scan row starting point for each scan row, the scan row ending point for each scan row, and the first set ending point for the first set of ultrasound images.

11. The system of claim 8, wherein the system controller is configured to control the electromechanical positioning apparatus, using the optical data, to control a distance between the scan head and the cellular tissue.

12. A system for screening and diagnostics of cellular tissue, the system comprising:
an ultrasound apparatus comprising a scan head;
an electromechanical positioning apparatus comprising an articulated arm and an optical sensor, the scan head and the optical sensor coupled to a free end of the articulated arm;
a system controller communicably coupled to the ultrasound apparatus and to the electromechanical positioning apparatus, the system controller being configured to control the electromechanical positioning apparatus, using optical data received from the optical sensor, to move the scan head adjacent the cellular tissue while controlling the ultrasound apparatus to generate a first set of ultrasound images of the cellular tissue; and
an image analyzer communicably coupled to the ultrasound apparatus to receive ultrasound images and to the system controller, wherein in response to receiving each ultrasound image of the first set of ultrasound images, the image analyzer is configured to analyze each received ultrasound image to determine whether each ultrasound image is conforming with predetermined parameters, to generate image optimization data indicating whether each ultrasound image is conforming with predetermined parameters, and to communicate the image optimization data to the system controller;
wherein in response to receiving the image optimization data indicating that one of the ultrasound images does not conform to the predetermined parameters, the system controller is configured to perform at least one of: control the electromechanical positioning apparatus to adjust a position of the scan head with respect to the cellular tissue and control the ultrasound apparatus to adjust ultrasound image acquisition parameters.

13. The system of claim 12, wherein the image analyzer comprises a machine learning module trained for ultrasound image analysis, the image analyzer being configured to analyze each received ultrasound image and to generate the image optimization data.

14. The system of claim 12, wherein adjusting ultrasound image acquisition parameters comprises adjusting an image acquisition depth.

15. The system of claim 12, wherein adjusting the position of the scan head with respect to the cellular tissue comprises adjusting a pressure exerted by the scan head on the cellular tissue.

16. A method for screening and diagnostics of cellular tissue, the method comprising:
controlling, using a system controller, an electromechanical positioning apparatus and an ultrasound apparatus to move an articulated arm of the electromechanical positioning apparatus and to move a scan head of the ultrasound apparatus adjacent the cellular tissue, the scan head coupled to a free end of the articulated arm;
controlling, using the system controller while moving the scan head adjacent the cellular tissue, the ultrasound apparatus to generate a first set of ultrasound images of the cellular tissue;
analyzing, using an image analyzer, each ultrasound image of the first set of ultrasound images to identify a potential abnormality in the cellular tissue and to determine a location of the potential abnormality within the cellular tissue;
generating, using the image analyzer, potential abnormality data comprising one of the location of the potential abnormality within the cellular tissue or an indicator that no abnormality is identifiable within the first set of ultrasound images;
communicating, using the image analyzer, the potential abnormality data to the system controller; and
controlling, using the system controller and in response to receiving the potential abnormality data including the location of the potential abnormality, the electromechanical positioning apparatus to move the scan head to a first position adjacent the location of the potential abnormality within the cellular tissue; and
controlling, using the system controller and starting from the first position, at least one of the electromechanical positioning apparatus and the ultrasound apparatus to generate a second set of ultrasound images of the potential abnormality, the second set of ultrasound images and the first set of ultrasound images detailing different aspects of the potential abnormality.

17. The method of claim 16, wherein the articulated arm is configured to move the scan head in six independent degrees of freedom.

18. The method of claim 16, wherein generating the second set of ultrasound images comprises at least one of:
controlling the electromechanical positioning apparatus to rotate the scan head about a z-axis passing through the potential abnormality;
controlling the ultrasound apparatus to operate in a shear-wave elastography mode;
controlling the ultrasound apparatus to operate in a Doppler mode;
controlling the ultrasound apparatus to operate in a mode without spatial compounding imaging; and
controlling the ultrasound apparatus to operate in a mode using harmonic imaging.

19. The method of claim 16 further comprising, following generation of the second set of ultrasound images, controlling, using the system controller, at least one of the electromechanical positioning apparatus and the ultrasound apparatus to generate a third set of ultrasound images of the potential abnormality, the third set of ultrasound images, the second set of ultrasound images, and the first set of ultrasound images each detailing different aspects of the potential abnormality.

20. The method of claim 16, wherein the first set of ultrasound images comprises a plurality of ultrasound images generated across two or more scan rows.

21. The method of claim 16, the image analyzer comprising a machine learning module trained for ultrasound image analysis, wherein analyzing each ultrasound image of the first set of ultrasound images comprises analyzing each ultrasound image of the first set of ultrasound images using the machine learning module.

22. The method of claim 21, wherein analyzing each received ultrasound image comprises analyzing each received ultrasound image, using the machine learning module, in conjunction with at least one of one or more sequentially prior generated ultrasound images and one or more sequentially subsequent generated ultrasound images.

23. The method of claim 16, further comprising receiving, using the system controller, optical data generated by an optical sensor coupled to the free end of the articulated arm.

24. The method of claim 23, the first set of ultrasound images being generated across two or more scan rows, further comprising determining, from the optical data using the system controller, a first set starting point for the first set of ultrasound images, a scan row starting point for each scan row, a scan row ending point for each scan row, and a first set ending point for the first set of ultrasound images.

25. The method of claim 24, further comprising generating, using the optical sensor, the optical data using a plurality of visual markers disposed on or adjacent the cellular tissue, the plurality of visual markers representing the first set starting point for the first set of ultrasound images, the scan row starting point for each scan row, the scan row ending point for each scan row, and the first set ending point for the first set of ultrasound images.

26. The method of claim 23, further comprising controlling, using the system controller and the optical sensor, the electromechanical positioning apparatus to control a distance between the scan head and the cellular tissue.

27. A method for screening and diagnostics of cellular tissue, the method comprising:
controlling, using a system controller, an electromechanical positioning apparatus and an ultrasound apparatus to move an articulated arm of the electromechanical positioning apparatus and to move a scan head of the ultrasound apparatus adjacent the cellular tissue, the scan head coupled to a free end of the articulated arm;
controlling, using the system controller while moving the scan head adjacent the cellular tissue, the ultrasound apparatus to generate a first set of ultrasound images of the cellular tissue;
analyzing, using an image analyzer, each ultrasound image of the first set of ultrasound images to determine whether each ultrasound image conforms with predetermined parameters;
generating, using the image analyzer, image optimization data indicating whether each ultrasound image conforms with the predetermined parameters;
communicating, using the image analyzer, the image optimization data to the system controller; and
controlling, using the system controller in response to receiving the image optimization data indicating that at least one of the ultrasound images does not conform with the predetermined parameters, at least one of: the electromechanical positioning apparatus, to adjust a position of the scan head with respect to the cellular tissue, and the ultrasound apparatus to adjust ultrasound image acquisition parameters.

28. The method of claim 27, the image analyzer comprising a machine learning module trained for ultrasound image analysis, wherein analyzing the each ultrasound image of the first set of ultrasound images comprises analyzing each ultrasound image of the first set of ultrasound images using the machine learning module.

29. The method of claim 27, wherein adjusting ultrasound image acquisition parameters comprises adjusting an image acquisition depth.

30. The method of claim 27, wherein adjusting the position of the scan head with respect to the cellular tissue comprises adjusting a pressure exerted by the scan head on the cellular tissue.
